(19) United States
(12) Patent Application Publication
(10) Pub. No.: US 2009/0082597 A1
(43) Pub. Date: Mar. 26, 2009

(54) PROCESS FOR PREPARING A COMPOUND

(75) Inventors: Arne Grumann, Kauniainen (FI); Kari Lappalainen, Espoo (FI); Peter Maiwald, Helsinki (FI); Pekka Pietikainen, Espoo (FI); Petteri Rummakko, Espoo (FI)

Correspondence Address: BIRCH STEWART KOLASCH & BIRCH, PO BOX 747, FALLS CHURCH, VA, 22040-0747, US

(73) Assignee: FERMION OY, Espoo (FI)

(21) Appl. No.: 11/988,796

(22) PCT Filed: Jul. 14, 2006
(86) PCT No.: PCT/FI2006/000252

§ 371 Date: Mar. 26, 2008

Related U.S. Application Data
(60) Provisional application No. 60/699,502, filed on Jul. 15, 2005.

(30) Foreign Application Priority Data
Jul. 15, 2005 (FI) 20055413

(51) Int. Cl. C07C 213/06

(52) U.S. Cl. 564/353

(57) ABSTRACT

The invention relates to the use of copper-catalyzed nucleophilic aromatic substitution reaction for preparing 3-aryloxy-3-arylpropylamines and more specifically to a method of preparing certain 3-aryloxy-3-arylpropylamines and the pharmaceutically acceptable addition salts thereof, comprising reacting an amino alcohol with a haloaromatic compound in the presence of a base and a catalytic copper source, and in the absence of a separate ligand.

### FIELD OF THE INVENTION

The present invention relates to a method of preparing 3-aryloxy-3-arylpropylamines and more particularly to a method of preparing a compound of Formula I

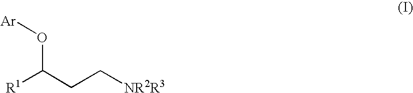

wherein
Ar is phenyl, substituted phenyl, heteroaryl, substituted heteroaryl, naphthyl, or substituted naphthyl;
R¹ is alkyl, phenyl, substituted phenyl, heteroaryl, substituted heteroaryl or alkenyl;
R² is hydrogen, alkyl, phenyl, substituted phenyl, heteroaryl, substituted heteroaryl, alkenyl, acyl, alkylO₂C—, heteroalkylO₂C—, arylO₂C— or heteroarylO₂C—;
R³ is hydrogen, alkyl, phenyl, substituted phenyl, heteroaryl, substituted heteroaryl or alkenyl;
and the pharmaceutically acceptable addition salts thereof.

The present invention further concerns the use of enantiomerically pure (R)-3-hydroxy-N-methyl-3-phenyl-propylamine for the preparation of atomoxetine.

### BACKGROUND OF THE INVENTION

Certain 3-aryloxy-3-arylpropylamines, including atomoxetine, are known to have central nervous system activity. Atomoxetine hydrochloride was previously named tomoxetine hydrochloride. (R)-Tomoxetine is a radioligand that binds to the norepinephrine uptake site with high affinity and it has been used as norepinephrine reuptake inhibitor in the treatment of attention deficit hyperactivity disorder (ADHD).

Several syntheses for the preparation of 3-aryloxy-3-arylpropylamines are known in the art. Also different methods for the resolution of racemic mixtures of 3-aryloxy-3-phenylpropylamines as well as 3-phenyl-3-hydroxypropylamines are known.

U.S. Pat. No. 4,314,081 discloses 3-Aryloxy-3-phenylpropylamines and acid additions salts thereof, which are useful as psychotropic agents, particularly as anti-depressants. The disclosed synthesis of atomoxetine comprises reaction of racemic 2-bromobenzylic compound with ortho-cresol, and the final step of this method is optical resolution of racemic atomoxetine.

U.S. Pat. No. 4,777,291 discloses a process for the epimerization of (+)-N-methyl-3-(2-methylphenoxy)-3-phenylpropylamine to its racemic form with an anion forming compound in a suitable solvent. In the optical resolution, 50% of the material, the (S)-tomoxetine enantiomer, is lost. The (S)-Isomer is racemiced with a strong base to give racemic atomoxetine, which is used in the optical resolution step again.

DE 4123253 A1 discloses a method of enzymatically hydrolyzing racemic ester(s) of halogenated aryl-alkanol(s) to give pure (R)-alcohol and pure (S)-ester. The preparation of enantiomerically pure (R)-alcohols of and/or enantiomerically pure (S)-esters comprises reacting racemic mixtures of esters with a hydrolase in the pH range 5-9 and separating the pure enantiomers. The pure (R)-alcohol and pure (S)-ester can then be further reacted to tomoxetine, fluoxetine and nisoxetine by direct substitution or under the conditions of Mitsunobu inversion to give the corresponding aryl ether, followed by replacement of the halogen by substitution with methylamine, followed by reaction with HCl.

Also U.S. Pat. No. 4,868,344 discloses the use of Mitsunobu reaction for synthesis of (R)-Atomoxetine. The disadvantages of this method are phosphine containing waste, which is a big problem on large scale. It is hard to remove and in addition the limits of P-compounds in wastewater are low. Also toxic chemicals are used in the Mitsunobu reaction (“Diethylazo dicarboxylate, DEAD).

WO 00/61540 discloses a method of preparing 3-aryloxy-3-arylpropylamines by nucleophilic aromatic displacement using complex benzylic alcohols, such as N-methyl-3-phenyl-3-hydroxypropylamine, with unactivated aromatics in 1,3-dimethyl-2-imidazolidione or N-methylpyrrolidinone. The starting material is always a racemic amino alcohol. The reaction comprises a nucleophilic aromatic displacement of 2-fluorotoluene with an alkoxide of a benzylic alcohol (20 h at 110° C. in toluene) and subsequently optical resolution of racemic product. In this method 3 equivalents of 2-fluorotoluene is used.

A method of preparing enantiomerically pure norfluoxetine, fluoxetine and tomoxetine is disclosed by Thomas M Koenig et al. Tetrahedron Letters, Vol 35, No 9. (1994) pp. 1339-1342. All final products in this article are derived from 3-phenyl-3-hydroxypropylamine intermediate. Treatment of the alkoxide of (S)-N-methyl-3-phenyl-3-hydroxypropylamine with 2-fluorotoluene and subsequent salt formation gave S-tomoxetine hydrochloride, however, epimerization of the chiral center was observed.

WO 00/58262 discloses a stereospecific processes for the preparation of tomoxetine using a nucleophilic aromatic displacement of activated ortho-substituted aromatic compound. Herein a chiral alcohol is used as starting material. The key reaction is activating the ortho-substituent (eg. formyl or imino, tert-butylimino), which has to be converted to ortho-methyl group in 5 or 6 steps long route with low overall yield.

Problems with the known methods of preparing 3-aryloxy-3-arylpropylamines are the low selectivity of the reaction products and epimerization of the chiral center during the reaction. Thus, the above processes do not resolve the problem of avoiding the production of undesired enantiomers in an efficient manner.

The present invention seeks to overcome the problems of the methods described above by providing a method of preparing 3-aryloxy-3-arylpropylamines which can be used to increase the selectivity and which can preferably lower the production costs. In the present invention an Ullmann-type reaction is utilized.

There are two different transformations referred as the Ullmann Reaction. The “classic” Ullmann Reaction is the synthesis of symmetric biaryls via copper-catalyzed coupling. The term “Ullmann-type reaction” refers to reactions that include copper-catalyzed nucleophilic aromatic substitution between various nucleophiles with aryl halides. The most common of these is the Ullmann ether synthesis.

A general procedure using catalytic amount of a copper complex for the formation of diaryl ethers from the reaction of aryl bromides and iodides with a variety of phenols was reported by Buchwald (Marcoux, J. F.; Doye, S.; Buchwald, S. L. J. Am. Chem. Soc. 1997, 119, 10539).

WO 02/085838 discloses copper-catalyzed carbon-heteroatom and carbon-carbon bond-forming methods, including copper-catalyzed methods of forming a carbon-oxygen bond between the oxygen atom of an alcohol and the activated carbon of an aryl, heteroaryl, or vinyl halide or sulfonate in the presence of a catalytic copper source, a ligand and a base. In the methods disclosed in WO 02/085838 a catalyst comprising a copper atom or ion and a ligand is always used. The methods disclosed do not relate to the production of atomoxetine.

### SUMMARY OF THE INVENTION

An object of the present invention is thus to provide a method so as to overcome the above problems. The objects of the invention are achieved by a method and use, which are characterized by what is stated in the independent claims. The preferred embodiments of the invention are disclosed in the dependent claims.

Accordingly the present invention provides as a first aspect a method of preparing a compound of Formula I

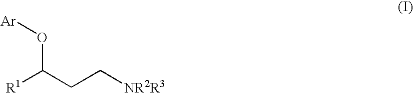

wherein
Ar is phenyl, substituted phenyl, heteroaryl, substituted heteroaryl, naphthyl, or substituted naphthyl;
R¹ is alkyl, phenyl, substituted phenyl, heteroaryl, substituted heteroaryl or alkenyl;
R² is hydrogen, alkyl, phenyl, substituted phenyl, heteroaryl, substituted heteroaryl, alkenyl, acyl, alkylO₂C—, heteroalkylO₂C—, arylO₂C— or heteroarylO₂C—;
R³ is hydrogen, alkyl, phenyl, substituted phenyl, heteroaryl, substituted heteroaryl or alkenyl;
and the pharmaceutically acceptable addition salts thereof, comprising the steps of:

a) reacting a compound of Formula II

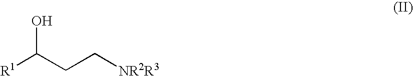

wherein R¹, R² and R³ are as defined above,
with a haloaromatic compound of Formula III

Ar—X (III)

wherein Ar is as defined above and X is a leaving group such as halogen, alkylsulfonate or arylsulfonate, in the presence of a base and a catalytic copper source, and in the absence of a separate ligand; and

b) optional formation of an acid addition salt using a suitable acid.

Preferably the method comprises resolution of the compound of Formula II before step a) or resolution of the obtained compound of Formula I.

In a second aspect the invention provides the use of enantiomerically pure (R)-3-hydroxy-N-methyl-3-phenyl-propylamine for the preparation of atomoxetine by copper-catalyzed nucleophilic aromatic substitution.

The invention is based on the realization that the use of copper-catalyzed nucleophilic aromatic substitution reaction (Ullmann type reaction) for preparing 3-aryloxy-3-arylpropylamines is very efficient and selective, especially when preparing atomoxetine.

It is an advantage of the method of the invention that enantiomerically pure products can be obtained as no racemisation occurs in the reaction. Therefore enantiomerically pure starting material can be used and early resolution of the starting material utilized, which is economically favorable over resolution of the final product. This means that half the amount of starting materials is needed resulting in a more environmentally friendly process using reduced amounts of solvents and other reagents. That means that chemicals are not wasted on the synthesis of the unwanted enantiomer, which has to be removed on the very last step It has also been noticed that cheaper bases, like K₃PO₄ or K₂CO₃, or their mixtures, can be used instead of Cs₂CO₃.

It is also an advantage of the method of this invention that only 1 or 1.1 equivalent of haloaromatic compound (e.g. 2-iodotoluene) is sufficient in the reaction. Thus, the method of the invention uses preferably only ⅙ of the amount of the haloaromatic compound compared to the known methods.

### DETAILED DESCRIPTION OF THE INVENTION

As will be appreciated by the skilled artisan, the present methods are not necessarily limited to the preparation of a specific isomer. Rather the present methods are capable of preparing either of the specific enantiomers or racemic mixtures depending on the enantiomeric purity of the starting materials used. The present invention is most useful as a preparation method of substantially pure atomoxetine, (R)-3-hydroxy-N-methyl-3-phenylpropylamine, utilizing a starting enantiomerically pure alcohol.

The term “stereoisomers” refers to compounds which have identical chemical constitution, but differ with regard to the arrangement of their atoms or groups in space. In particular, the term “enantiomers” refers to two stereoisomers of a compound which are non-superimposable mirror images of one another.

The term “epimerization” refers to a process in which the configuration about one chiral centre of a compound is inverted to give the opposite configuration. The term “chiral” refers to molecules which have the property of nonsuperimposability on their mirror image.

The term “ee” or “enantiomeric excess” refers to the percent by which one enantiomer, El, is in excess in a mixture of both enantiomers (E1+E2), as calculated by the equation {(E1−E2)/(E1+E2)}×100%=ee. The present invention relates to processes for the preparation of 3-aryloxy-3-arylpropylamines. It is understood by the skilled person that these compounds exist as stereoisomers. Herein, the Cahn-Prelog-Ingold designations of (R)-and (S)-are used to refer to specific isomers where designated. Specifically, present invention relates to processes for the preparation of (R)-atomoxetine, (R)-N-methyl-3-(2-methylphenoxy)-benzenepropanamine.

The term enantiomerically enriched refers to a chiral substance whose enentiomeric ratio is greater than 50:50 but less than 100:0.

As used herein the term “substantially pure” refers to enantiomeric purity of the compounds. The specific isomers can be obtained by resolution of the starting materials, intermediates, or in some cases the product. For example, atomoxetine specific isomers can be most conveniently obtained by utilizing enantiomerically pure starting materials, specifically, (R)-3-hydroxy-N-methyl-3-phenylpropylamine. As used herein the term “enantiomerically pure” refers to an enantiomeric excess which is higher than 90%, preferably higher than 95%, more preferably higher than 99% and most preferably 99.8% or even higher.

The substantially pure isomers of the starting alcohols can be obtained by stereospecific reduction or resolved and recovered by techniques known in the art, such as, chromatography on chiral stationary phases and fractional recrystallization of addition salts formed by reagents used for that purpose. Useful methods of resolving and recovering specific stereoisomers are known in the art.

The catalytic copper source used in the method does not comprise a separate ligand. The phrase “in the absence of a separate ligand” means that there is not an effective amount of a separate ligand present in the reaction.

The term “pharmaceutically acceptable addition salt” refers to an acid addition salt. The 3-aryloxy-3-arylpropylamines described herein form pharmaceutically acceptable addition salts with a wide variety of organic and inorganic acids and include the physiologically acceptable salts which are often used in pharmaceutical chemistry. A pharmaceutically acceptable addition salt is formed from a suitable acid as is well known in the art. Such salts are also part of this invention.

Typical inorganic acids used to form such salts include hydrochloric, hydrobromic, hydriodic, nitric, sulfuric, phosphoric, hypophosphoric, metaphosphoric, pyrophosphoric, and the like acids. Salts derived from organic acids, such as aliphatic mono and dicarboxylic acids, phenyl substituted alkanoic acids, hydroxyalkanoic and hydroxyalkandioic acids, aromatic acids, aliphatic and aromatic sulfonic acids, may also be used. Such pharmaceutically acceptable salts thus include acetate, phenylacetate, trifluoroacetate, acrylate, ascorbate, benzoate, chlorobenzoate, dinitrobenzoate, hydroxybenzoate, methoxybenzoate, methylbenzoate, o-acetoxybenzoate, naphthalene-2-benzoate, bromide, isobutyrate, phenylbutyrate, P-hydroxybutyrate, butyne-1,4-dicarboxylate, hexyne-1,4-dicarboxylate, caprate, caprylate, cinnamate, citrate, formate, fumarate, glycollate, heptanoate, hippurate, lactate, malate, maleate, hydroxymaleate, malonate, mandelate, mesylate, nicotinate, isonicotinate, nitrate, oxalate, phthalate, teraphthalate, propiolate, propionate, phenylpropionate, salicylate, sebacate, succinate, suberate, benzene-sulfonate, p-bromobenzenesulfonate, chlorobenzenesulfonate, ethanesulfonate, 2-hydroxyethanesulfonate, methanesulfonate, naphthalene-1-sulfonate, naphthalene-2-sulfonate, p-toluenesulfonate, xylenesulfonate, tartarate, and the like.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In preferred embodiments, a straight chain or branched chain alkyl has 30 or fewer carbon atoms in its backbone (e.g. C₁-C₃₀ for straight chain, C₃-C₃₀ for branched chain), and more preferably 20 of fewer. Likewise, preferred cycloalkyls have from 4-10 carbon atoms in their ring structure, and more preferably have 5, 6 or 7 carbons in the ring structure. Alkyl can also be a “lower alkyl”, which as used herein means an alkyl group, as defined above, but having from one to ten carbons, more preferably from one to six carbon atoms in its backbone structure. Likewise, “lower alkenyl” and “lower alkynyl” have similar chain lengths.

The terms “alkenyl” and “alkynyl” refer to unsaturated aliphatic groups analogous in length and possible substitution to the alkyls described above, but which contain at least one double or triple carbon-carbon bond, respectively.

The term “aryl” as used herein includes 4-, 5-, 6-and 7-membered single-ring aromatic groups which may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “heteroaryl”. The aromatic ring can be substituted at one or more ring positions with such substituents as for example, halogens, alkyls, alkenyls, alkynyls, hydroxyl, amino, nitro, thiol, amines, imines, amides, phosphonates, phosphines, carbonyls, carboxyls, silyls, ethers, thioethers, sulfonyl, selenoethers, ketones, aldehydes, esters, or the like.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described hereinabove. The permissible substituents can be one or more and the same or different for appropriate organic compounds. For purposes of this invention, the heteroatoms such as nitrogen may have hydrogen substituents and/or any permissible substituents of organic compounds described herein which satisfy the valencies of the heteroatoms. This invention is not intended to be limited in any manner by the permissible substituents of organic compounds.

The present invention provides a method of preparing a compound of Formula I and the pharmaceutically acceptable addition salts thereof, by reacting a compound of Formula II with a haloaromatic compound of Formula III in the presence of a catalytic copper source and a base, and optionally forming an acid addition salt using a pharmaceutically acceptable acid. Compounds of Formula I or II may be enantiomerically pure or racemic, and they may be resolved by methods known in the art either before the reaction or after it.

In one embodiment of the invention the catalytic copper source comprises a copper atom or ion and the catalytic copper source is preferably a Cu(I)-catalyst, such as CuI, CuCl, Cu(I)triflate benzene-complex, CuBr or Cu₂O. The catalyst may be added in amounts of 0.01 mol-% to 100 mol-% calculated from the amount of the starting material. In one embodiment the catalyst is added in an amount of 1 mol-% to 50 mol-%, preferably 2 mol-% to 10 mol-% calculated from the amount of the compound of Formula II.

In one embodiment of the invention the base is selected from K₂CO₃, KHCO₃ K₃PO₄, Cs₂CO₃, NaOH, KOH and NaOtBu, and preferably the base is K₃PO₄ or K₂CO₃ or a mixture thereof.

In general, the subject reactions are carried out in a liquid reaction medium. However, the reactions may be run without addition of solvent. If the solvent is used, it may be, any suitable inert solvent, preferably one in which the reaction ingredients, including the catalyst, are substantially soluble. For example the reaction of a compound of Formula II with a haloaromatic compound of Formula III in the method of the present invention can be carried out in a suitable solvent, including aromatic hydrocarbons like toluene, xylenes, mesitylene, and cumene, acetonitrile, methylisobutyl ketone (MIBK), tetrahydrofuran (THF), dimethoxyethane (DME) and anisole. Aromatic hydrocarbons are preferred solvents. In one embodiment of the invention the haloaromatic compound of Formula III is ortho-iodotoluene.

The present invention is especially suitable for the preparation of atomoxetine

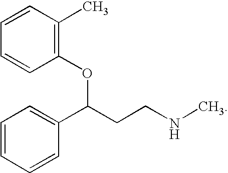

When atomoxetine is prepared, the compound of Formula II is 3-hydroxy-N-methyl-3-phenyl-propylamine, and it is preferably subjected to an optical resolution before step a) to obtain (R)-3-hydroxy-N-methyl-3-phenyl-propylamine, which can be used for the stereospecific preparation of atomoxetine or the racemic atomoxetine may be resolved after the reaction to obtain enantiometrically pure product. It is another embodiment of the invention to prepare (R)-atomoxetine

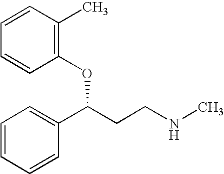

Thus, the method of the present invention comprises preferably the steps of:
a) resolution of 3-hydroxy-N-methyl-3-phenyl-propylamine to give enantiomerically enriched (R)-3-hydroxy-N-methyl-3-phenyl-propylamine;
b) reacting enantiomerically enriched (R)-3-hydroxy-N-methyl-3-phenyl-propylamine with ortho-halogenotoluene, preferably ortho-iodotoluene, in the presence of Cu(I)-catalyst, preferably copper(I)iodide, and a base, preferably K₃PO4, or K₂CO₃ or mixtures thereof, to give atomoxetine; and
c) optionally formation of a pharmaceutically acceptable acid addition salt using a suitable acid.

In one preferred embodiment of the invention (R)-3-hydroxy-N-methyl-3-phenyl-propylamine in steps a) and b) is enantiomerically pure.

In one preferred embodiment of the invention hydrochloric acid is used in step c) for the preparation of atomoxetine hydrochloride.

In one aspect of the invention the method further comprises forming of a pharmaceutical product from the compound of formula I or from the pharmaceutically acceptable addition salt thereof.

The methods of the present invention may be performed under a wide range of conditions, though it will be understood that the solvents and temperature ranges recited herein are not limiting and only correspond to a preferred mode of the process of the invention.

In general, it will be desirable that reactions are run using mild conditions which will not adversely affect the reactants, the catalyst, or the product. For example, the reaction temperature influences the speed of the reaction, as well as the stability of the reactants, products and catalyst.

In certain embodiments, the methods of the present invention are conducted at a temperature less than about 170° C., less than about 150° C., less than about 110° C., less than about 100° C., less than about 90° C., less than about 50° C. or less than about 40° C. and in certain embodiments, the methods of the present invention are conducted at ambient temperature.

### EXAMPLES

### Example 1

### Preparation of (R)-N-Methyl-3-(2-Methylphenoxy)-Benzenepropanamine Hydrochloride

(3R)-methyl-3-hydroxy-3-phenylpropylamine-(S)-mandelate salt:

A 5 L vessel was charged with 165,2 g N-methyl-3-hydroxy-3-phenylpropylamine and 68.5 g (S)-(+)-mandelic acid. 3300 ml ethyl acetate was added and the clear solution heated to 50° C. for 30 min. The mixture was then slowly cooled to 20° C. and stirred for 12 h at this temperature. Filtration of the suspension followed by drying under reduced pressure at 50° C. over night gave 107.5 g (75%) of (3R)-methyl-3-hydroxy-3-phenylpropylamine-(S)-mandelate salt with an enantiomeric excess of 83% as determined by chiral HPLC analysis.

A 3 L reaction vessel was charged with 105 g of the above-mentioned (3R)-methyl-3-hydroxy-3-phenylpropylamine-(S)-mandelate, 1340 ml of acetone and 420 ml of MTBE. The mixture was heated to 50° C. causing all solids to dissolve. Upon slow cooling to room temperature and continued stirring for 12 h, 82 g of (3R)-methyl-3-hydroxy-3-phenylpropylamine-(S)-mandelate with an enantiomeric purity of 99.5% was obtained after drying at reduced pressure at about 50° C. over night.

(3R)-methyl-3-hydroxy-3-phenylpropylamine:

81 g of the (3R)-methyl-3-hydroxy-3-phenylpropylamine-(S)-mandelate was suspended in 300 ml of toluene followed by addition of 200 ml of 3 M NaOH. The mixture was stirred for 30 min after which all solids had disappeared. The phases were separated and the aqueous phase was extracted with 100 ml of toluene. The toluene phases were combined and evaporated under reduced pressure giving 40.0 g of (3R)-methyl-3-hydroxy-3-phenylpropylamine with an enantiomeric excess of >99% as determined by chiral HPLC.

(R)-N-methyl-3-(2-methylphenoxy)-benzenepropanamine hydrochloride:

A 3-necked 100 ml glass reactor was flushed for 15 min with N₂ and subsequently charged with 15 g (90.8 mmol) of the above mentioned (3R)-methyl-3-hydroxy-3-phenylpropylamine (>99% ee, chiral HPLC), potassium phosphate (28.9 g, 136.2 mmol) and 1.73 g copper(I)iodide (9.8 mmol, 10 mol-%). 60 ml of toluene was added to the mixture and the suspension was stirred for 5 min. 12.8 ml (100 mmol) of 2-iodotoluene was added and the reaction mixture was heated to reflux for 24 h. After cooling to room temperature, the suspension was filtered and the filter cake was washed with 60 ml of toluene. 75 ml of water was added to the filtrate and the mixture was stirred for 10 min at room temperature. The aqueous phase was brought to pH 1-2 with 30% HCl and the phases were separated. 60 ml of toluene was added to the aqueous phase and aqueous NaOH was added until pH 12-14 of the aqueous phase was reached. After stirring for 10 min the phases were separated. The organic phase was evaporated under reduced pressure yielding 25 g of an oil.

The oil was redissolved in 80 ml of toluene, warmed to 80° C. and 36 g of a 10% HCl-ethyl acetate solution was added dropwise to the solution. During cooling of the solution a white solid precipitated. After 5 h at room temperature, the suspension was filtered and the residue was dried in vacuum at about 50° C. to yield 22 g (75.4 mmol, 83%) of (R)-N-methyl-3-(2-methylphenoxy)-benzenepropanamine hydrochloride.

The (R)-N-methyl-3-(2-methylphenoxy)-benzenepropanamine hydrochloride salt was placed in a 100 ml reaction vessel and 55 ml of isopropanol was added. Upon heating to reflux temperature all solids were dissolved. Slow cooling to room temperature gave 18.1 g (82%) of colorless (R)-N-methyl-3-(2-methylphenoxy)-benzenepropanamine hydrochloride (>99% ee, HPLC).

### Example 2

### Preparation of N-Methyl-3-(2-Methylphenoxy-Benzenepropanamine Hydrochloride

A 100 ml flask was flushed for 15 min with N₂ and subsequently charged with 10 g (60.5 mmol) N-methyl-3-hydroxy-3-phenylpropylamine, 15,3 g (72 mmol) potassium phosphate and 1.14 g copper iodide (6.0 mmol, 10 mol-%). 40 ml of toluene followed by 7.7 ml (60 mmol) of 2-iodotoluene were added to the mixture and the suspension was heated to reflux for 20 h. After cooling to room temperature, the suspension was filtered and the residue was washed with 20 ml of toluene. 30 ml of water was added to the filtrate and the mixture was stirred for 15 min at room temperature. The phases were separated and 30 ml of water was added to the toluene phase. The aqueous phase was brought to pH 1 with 30% HCl. The phases were stirred and separated. The aqueous phase was brought to pH 12 with aqueous NaOH followed by addition of 30 ml toluene. The mixture was heated to 50° C. and the phases were separated. The toluene phase was evaporated giving 7.4 g of an oil.

5,8 g of the residue was dissolved in 18 ml of toluene, warmed to 80° C. and 11,1 g of 7.7% HCl-ethyl acetate solution was added dropwise to the solution. After 15 min stirring at reflux temperature the solution was cooled to 0° C. with a rate of 10-15° C./h. The precipitate was collected and dried under reduced pressure at 40° C. over night. Yield 5.0 g (17.1 mmol, 75%).

The crystals obtained in the before mentioned crystallization were combined with 17 ml of toluene and heated to reflux, causing all solids to dissolve. Upon cooling to room temperature, 4,3 g (86%) of N-methyl-3-(2-methylphenoxy)-benzenepropanamine hydrochloride was collected after filtration and drying at about 50° C. under reduced pressure.

### Example 3

### Preparation of N-Methyl-3-(2-Methylphenoxy-Benzenepropanamine Hydrochloride

A 10 ml flask was subsequently filled with 1 g (6.1 mmol) N-methyl-3-hydroxy-3-phenylpropylamine, 2.6 g (12.2 mmol) potassium phosphate and 0.11 g copper iodide (0.6 mmol, 10 mol-%) under a flow of nitrogen. 15 ml of acetonitrile and 1.17 g 2-iodotoluene (9.2 mmol) were added to the mixture and the suspension was heated to reflux temperature. After heating for about 30 h the mixture was cooled to room temperature. The mixture was filtrated and the residue washed with 15 ml acetonitrile. The organic phase was evaporated and redissolved in 30 ml toluene. 15 ml water was added and the aqueous phase was brought to pH 1 with 30% aq. HCl. The phases were separated and the aqueous phase was brought to pH 12 with aqueous KOH. 10 ml of toluene was added and the mixture was stirred for 15 min, after which the phases were separated. The combined toluene phases were evaporated giving 1.6 g of an oil.

The oil was dissolved in 5 ml of toluene and heated to reflux temperature. 1,85 g of 12%-HCl-ethyl acetate was added and the solution was cooled to room temperature. After filtration and drying of the residue under reduced pressure at 50° C. 1.3 g (4.5 mmol, 71%) of N-methyl-3-(2-methylphenoxy)-benzenepropanamine hydrochloride was collected.

Recrystallization of the received solids from 4 ml of MIBK gave 1.1 g (85%) of N-methyl-3-(2-methylphenoxy)-benzenepropanamine hydrochloride after drying under reduced pressure at 50° C.

### Example 4

### Preparation of N-Methyl-3-(2-Methylphenoxy)-Benzenepropanamine Hydrochloride

A 100 ml flask was flushed for 15 min with N₂ and subsequently charged with 10 g (60.5 mmol) N-methyl-3-hydroxy-3-phenylpropylamine, 15,3 g (72 mmol) potassium phosphate and 604 mg copper(I)chloride (6.0 mmol, 10 mol-%). 40 ml of toluene followed by 8.4 ml (66 mmol) of 2-iodotoluene was added to the mixture and the suspension was heated to reflux for 26 h. After cooling to room temperature, the suspension was filtered and the residue washed with 20 ml of toluene. 30 ml of water was added to the filtrate and the mixture was stirred for 15 min at room temperature. The phases were separated and 30 ml of water was added to the toluene phase. The aqueous phase was brought to pH 1 with 30% HCl. The mixture was stirred and the phases were separated. The aqueous phase was brought to pH 12 with aqueous NaOH followed by addition of 30ml toluene. The mixture was heated to 50° C. and the phases were separated. The toluene phase was evaporated giving 7.4 g of an oil.

5,8 g of the residue was dissolved in 18ml of toluene, warmed to 80° C. and 11,1 g of 7.7% HCl-ethyl acetate solution was added dropwise to the solution. After 15 min stirring at reflux temperature the solution was cooled to 0° C. with a rate of 10-15° C./h. The precipitate was collected and dried under reduced pressure at 40° C. over night. Yield 5.0 g (17.1 mmol, 75%).

The crystals obtained in the before mentioned crystallization were combined with 17 ml of toluene and heated to reflux, causing all solids to dissolve. Upon cooling to room temperature, 4,3 g (86%) of N-methyl-3-(2-methylphenoxy)-benzenepropanamine hydrochloride was collected after filtration and drying at about 50° C. under reduced pressure.

### Example 5

### Preparation of (R)-N-Methyl-3-(2-Methylphenoxy)-Benzenepropanamine Hydrochloride

A 3L vessel was charged with 165.2 g N-methyl-3-hydroxy-3-phenylpropylamine and 68.5 g (S)-(+)-mandelic acid. 1240 ml methyl acetate was added and the clear solution was heated to 50° C. for 30 min. The mixture was then cooled to 10° C. and stirred for 2 h at this temperature. Filtration of the suspension followed by drying under reduced pressure at 50° C. over night gave 108.7 g (76%) of (3R)-methyl-3-hydroxy-3- phenylpropylamine-(S)-mandelate salt with an enantiomeric excess of 94% as determined by chiral HPLC analysis.

60 g of the (3R)-methyl-3-hydroxy-3-phenylpropylamine-(S)-mandelate was suspended in 185 ml of cumene followed by addition of 50 ml of water and 16.5 g of 50% NaOH. The mixture was stirred for 30 min at 90° C. after which all solids had disappeared. The phases were separated and the aqueous phase was extracted with 185 ml of cumene at 90° C. The cumene phases were combined and 250 ml of cumene was evaporated under reduced pressure to give 119 g of a solution that was analyzed to contain 28.5 g (23.9 w-%) of (3R)-methyl-3-hydroxy-3-phenylpropylamine.

A 3-necked 250 ml glass reactor was flushed for 15 min with N₂ and subsequently charged with 119 g of the above mentioned (3R)-methyl-3-hydroxy-3-phenylpropylamine solution, potassium carbonate (50 g, 362 mmol) and 1.8 g copper(I)iodide (9.5 mmol, 5.5 mol-%). The suspension was stirred for 5 min, 26 ml (204 mmol) of 2-iodotoluene was added and the reaction mixture was heated to 148° C. for 21 h. After cooling to room temperature, the suspension was filtered and the filter cake was washed twice with 75 ml of toluene. 285 ml of water was added to the filtrate and the mixture was stirred for 10 min at 50° C. The aqueous phase was brought to pH 1-2 with 30% HCl and the phases were separated at 50° C. The organic phase was extracted with 100 ml water at 50° C. The aqueous phases were brought to pH 11-12 with aqueous 50% NaOH. After stirring for 15 min the phases were separated. 100 ml of toluene was distilled off under reduced pressure yielding 156 g of a solution that was analyzed to contain 34.8 g (22.3 w-%) of atomoxetine base.

A 3-necked 250 ml flask was charged with 53 g of the above mentioned atomoxetine base solution, warmed to 70° C. and 28.5 g of 7.4% HCl-ethyl acetate solution (57.8 mmol of HCl) was added dropwise to the solution. After 15 min stirring at reflux temperature the solution was cooled to 0° C. with a rate of 15-20° C./h. The precipitate was collected, washed with cold isopropanol and dried under reduced pressure at 40° C. over night. Yield 13.2 g (45.2 mmol, 98%). The enantiomeric excess of the product was >99% as determined by chiral HPLC.

Alternatively, a 3-necked 250 ml flask was charged with 53 g of the above mentioned atomoxetine base solution, warmed to 70° C. and 9.0 g of 23.4% HCl-isopropanol solution (57.7 mmol of HCl) was added dropwise to the solution. After 15 min stirring at reflux temperature the solution was cooled to 0° C. with a rate of 15-20° C./h. The precipitate was collected, washed with cold isopropanol and dried under reduced pressure at 40° C. over night. Yield 12.7 g (43.5 mmol, 94%). The enantiomeric excess of the product was >99% as determined by chiral HPLC.

1. A method of preparing a compound of Formula I 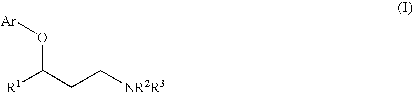
wherein
Ar is phenyl, substituted phenyl, heteroaryl, substituted heteroaryl, naphthyl, or substituted naphthyl;
R¹ is alkyl, phenyl, substituted phenyl, heteroaryl, substituted heteroaryl or alkenyl;
R² is hydrogen, alkyl, phenyl, substituted phenyl, heteroaryl, substituted heteroaryl, alkenyl, acyl, alkylO₂C—, heteroalkylO₂C—, arylO₂C— or heteroarylO₂C—;
R³ is hydrogen, alkyl, phenyl, substituted phenyl, heteroaryl, substituted heteroaryl or alkenyl; and the pharmaceutically acceptable addition salts thereof, comprising:
a) reacting a compound of Formula II 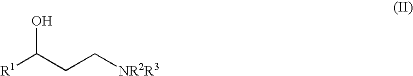
wherein R¹, R² and R³ are as defined above,
with a haloaromatic compound of Formula III
Ar-X (III)
wherein Ar is as defined above and X is a leaving group such as halogen, alkylsulfonate or arylsulfonate, in the presence of a base and a catalytic copper source, and in the absence of a separate ligand;
b) optionally preparing an acid addition salt using a suitable acid.

2. The method of claim 1 further comprising resolving the compound of Formula II before step a) or resolving the obtained compound of Formula I.

3. The method of claim 1, wherein the compound of formula II in step a) is optically active.

4. The method of claim 1 or 2 wherein the catalytic copper source comprises a copper atom or ion.

5. The method of claim 4, wherein the catalytic copper source is a Cu(I)-catalyst.

6. The method of claim 5, wherein the catalytic copper source is Cul, CuCl, Cu(I)triflate Benzene-complex, CuBr or Cu₂O.

7. The method of claim 1, wherein the catalyst is added in an amount of 1 mol-% to 50 mol-% calculated from the amount of the compound of Formula II.

8. The method of claim 7, wherein the catalyst is added in an amount of 2 mol-% to 10 mol-% calculated from the amount of the compound of Formula II.

9. The method of claim 1, wherein the base is selected from K₂CO₃, KHCO₃, K₃PO₄, Cs₂CO₃, NaOH, KOH and NaOtBu.

10. The method of claim 9, wherein the base is K₃PO₄ or K₂CO₃ or a mixture thereof.

11. The method of claim 1 , wherein the base is K₃PO₄ or K₂CO₃ or a mixture thereof and the catalytic copper source is Cul, CuCl, Cu(I)triflate Benzene-complex, CuBr or Cu₂O.

12. The method of claim 1, wherein the reaction is performed without the use of a solvent.

13. A method of claim 1, wherein the reaction is carried out in a solvent selected from aromatic hydrocarbons, acetonitrile, methylisobutyl ketone (MIBK), tetrahydrofuran (THF), anisole or dimethoxyethane (DME).

14. The method of claim 13, wherein an aromatic hydrocarbon is used as a solvent.

15. The method of claim 14 wherein the solvent is toluene, mesitylene, cumene, or xylene.

16. A method of claim 1, wherein the haloaromatic compound of Formula III is ortho-iodotoluene.

17. A method as claimed in claim 1, wherein the compound of Formula II is 3-hydroxy-N-methyl-3-phenyl-propylamine.

18. The method of claim 17, wherein the 3-hydroxy-N-methyl-3-phenyl-propylamine is subjected to an optical resolution before step a) to obtain (R)-3-Hydroxy-N-methyl-3-phenyl-propylamine.

19. A method as claimed in claim 1, wherein a compound of formula 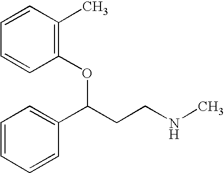
is prepared.

20. A method as claimed in claim 1, wherein a compound of formula 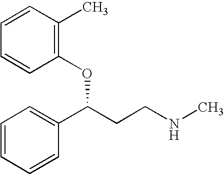
is prepared.

21. A method as claimed in claim 1, wherein atomoxetine hydrochloride is prepared.

22. A method of claim 1 comprising:
a) resolving of 3-hydroxy-N-methyl-3-phenyl-propylamine to give enantiomerically enriched (R)-3-hydroxy-N-methyl-3-phenyl-propylamine;
b) reacting enantiomerically enriched (R)-3-hydroxy-N-methyl-3-phenyl-propylamine with ortho-iodotoluene in the presence of Cu(I)-catalyst and a base to give atomoxetine; and
c) optionally preparing an acid addition salt using a suitable acid.

23. A method of claim 1 comprising:
a) resolving of 3-hydroxy-N-methyl-3-phenyl-propylamine to give enantiomerically pure (R)-3-hydroxy-N-methyl-3-phenyl-propylamine;
b) reacting enantiomerically pure (R)-3-hydroxy-N-methyl-3-phenyl-propylamine with ortho-iodotoluene in the presence of Cu(I)-catalyst and a base to give atomoxetine; and
c) optionally preparing an acid addition salt using a suitable acid.

24. Use of enantiomerically pure (R)-3-hydroxy-N-methyl-3-phenyl-propylamine for the preparation of atomoxetine by copper-catalyzed nucleophilic aromatic substitution.

25. Preparation of atomoxetine comprising:
a) reacting 3-hydroxy-N-methyl-3-phenyl-propylamine with ortho-halogenotoluene in the presence of a base and a catalytic copper source; and
b) optionally preparing an acid addition salt using a suitable acid.

26. The method of claim 25 further comprising resolving 3-hydroxy-N-methyl-3-phenyl-propylamine before step a) or resolving the obtained racemic atomoxetine.

27. The method of claim 25 where the base used is K₃PO₄ or K₂CO₃ or a mixture thereof.
